(19) United States
(12) Patent Application Publication
(10) Pub. No.: US 2017/0143834 A1
(43) Pub. Date: May 25, 2017

(54) FORMULATION FOR HGH AND RHIGF-1 COMBINATION

(71) Applicant: Ipsen Pharma S.A.S., Boulogne-Billancourt (FR)

(72) Inventors: Enona GOPINATH, Redwood City, CA (US); Susan PARK, San Francisco, CA (US); Tsutomu ARAKAWA, Thousand Oaks, CA (US); Joël RICHARD, Mere (FR); Fabio FAIS, Dreux (FR)

(21) Appl. No.: 15/281,361

(22) Filed: Sep. 30, 2016

Related U.S. Application Data
(63) Continuation of application No. 13/513,858, filed on Jun. 4, 2012, now abandoned.
(60) Provisional application No. 61/261,859, filed on Nov. 17, 2009.

(51) Int. Cl. A61K 47/18; A61K 47/10; A61K 47/02; A61K 47/26; A61K 38/30; A61K 38/27

(52) U.S. Cl. CPC A61K 47/183; A61K 38/30; A61K 38/27; A61K 47/02; A61K 47/26; A61K 47/10

(57) ABSTRACT

Pharmaceutical compositions, in particular, compositions comprising growth hormone (GH) and insulin-like growth factor (IGF-1) which are stable at a desirable pH without aggregation formation, and processes for preparing such compositions.

This application is a continuation of U.S. application Ser. No. 13/513,858, filed Jun. 4, 2012, which is the national phase of International Application No. PCT/EP2010/006996, filed Nov. 17, 2010, which claims the benefit of U.S. Provisional Patent Application No. 61/261,859, filed Nov. 17, 2009, the contents of each of which are hereby incorporated by reference in their entireties.

The present invention relates to pharmaceutical compositions. More particularly, the invention relates to formulations of growth hormone (GH) and insulin-like growth factor (IGF-1) combination compositions. These combination compositions provide stable liquid pharmaceutical compositions without the formation of visible insoluble aggregates at a desirable pH.

The present invention further provides a formulation for insulin-like growth factor 1 (IGF-1) and growth hormone (GH), wherein proteins may be formulated together in an injectable form, or formulated separately and mixed into a unit dosable injectable form prior to administration.

Insulin-like growth hormone belongs to the family of polypeptides known as somatomedins and is a polypeptide naturally occurring in human body fluids. Most tissues and especially the liver produces IGF-1 together with specific IGF-binding proteins. IGF-1 stimulates growth and division of a variety of cell types, particularly during development, thus processes such as skeletal growth and cell replication are affected by IGF-1 level. These molecules are under the control of growth hormone (GH).

IGF-1 is the primary protein hormone mediating the growth promoting effects of GH on bone. IGF-1 is produced in response to GH and then induces subsequent cellular responses, including cellular responses in bone. IGF-1 is composed of 70 amino acids in a single chain with three intramolecular disulfide bridges. IGF-1 has a molecular weight of 7649 daltons and is produced primarily by the liver as an endocrine hormone as well as in target tissues in a paracrine/autocrine fashion. IGF-1 has been manufactured recombinantly (rhIGF-1) on a large scale using both yeast and E. coli.

Growth hormone or human growth hormone (hGH) is a single-chain polypeptide consisting of 191 amino acids. Disulfide bonds link positions 53 and 165 and 182 and 189. Human GH is a potent anabolic agent. Among its most striking effects in hypopituitary (GH deficient) subjects is accelerated linear growth of bone growth plate cartilage resulting in increased stature.

The advantageous and synergic effect of the combination of both proteins is described in the international patent application WO9118621. Co-administration of IGF-1 and GH to a mammal gives rise to enhanced growth over the growth achieved using either IGF-1 or GH alone. The enhancement is equal to the sum of the growth observed when IGF-1 is administered and the growth observed when GH is administered.

Methods and compositions for increasing the growth rate are also disclosed in the international patent application WO 2006/130769. The study related essentially to a method of treatment and the results focused on the patient reaction. Pharmaceutical compositions are described and particularly a mixture of IGF-1 and GH formulated in mannitol, glycine and/or phosphate at pH 7.4. If the mixture is to be stored, it is formulated in a buffer such as citrate at a pH of about 6, with a surfactant that increases the solubility of the GH at this pH such as polysorbate 20 or poloxamer 188. It also describes the possibility of adding an inorganic salt and a stabilizer. No non-aggregating agent is used in the formulations disclosed in WO 2006/130769.

A problem frequently occurring when combining two proteins in a solution is the formation of complexes by protein-protein interactions. Such formation of complexes is particularly influenced by change in concentration, temperature, pH and buffer of the protein-containing solutions. The protein complexes may then form insoluble aggregates causing loss of potency and activity of the proteins.

Furthermore, in pharmaceutical formulations, the dosage of therapeutic protein is important and must be kept within controlled ranges over an extended period of time. The use of solubilizing agents is often required to obtain and maintain the right concentration of protein in solution and particularly to solubilize high amounts of proteins. U.S. Pat. No. 6,767,892 disclosed pharmaceutical compositions of IGF-1 and analogues thereof containing solubilizing compounds such as arginine, N-acetyl arginine or guanidine hydrochloride IGF-1. Compositions were tested, comparative data were provided with increased IGF-1 solubility at pH greater than 5.0 and at refrigerated temperatures. However this document does not disclose compositions comprising IGF-1 combined with further therapeutic proteins.

It is an object of the invention to prepare liquid formulations containing both IGF-1 and growth hormone (GH), which are stable at 4° C. for at least 30 days, with no significant aggregation as evidenced by visual clarity of the solution. A process for the preparation of a liquid formulation containing both IGF-1 and GH is a further object of the invention.

### DESCRIPTION OF FIGURES

FIG. 1: shows overlaid sedimentation velocity profiles obtained by analytical ultracentrifugation of a IGF-1 solution, GH solutions, and a 1:1 mixture of the two solutions. The first set of profiles (FIG. 1) was obtained with the proteins formulated in a 25 mM citrate buffer at pH 6, and shows evidence of substantial association between the proteins.

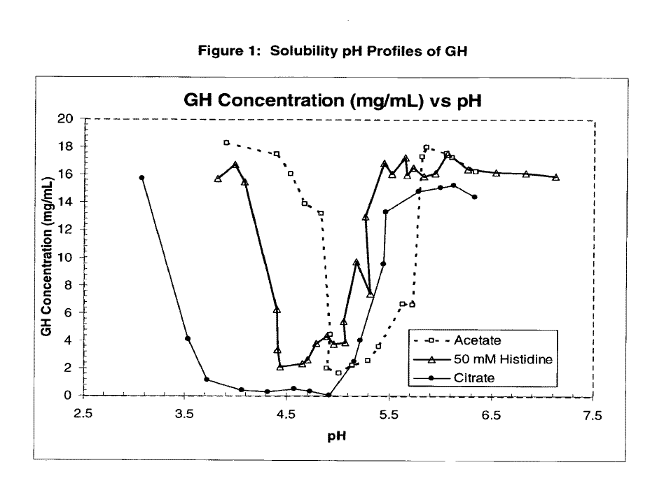

FIG. 2: shows sedimentation profiles of solutions including 100 mM argininium ion (arginine). The profiles show that the presence of arginine produces changes indicative of a reduced amount of high-molecular weight aggregates in the solutions.

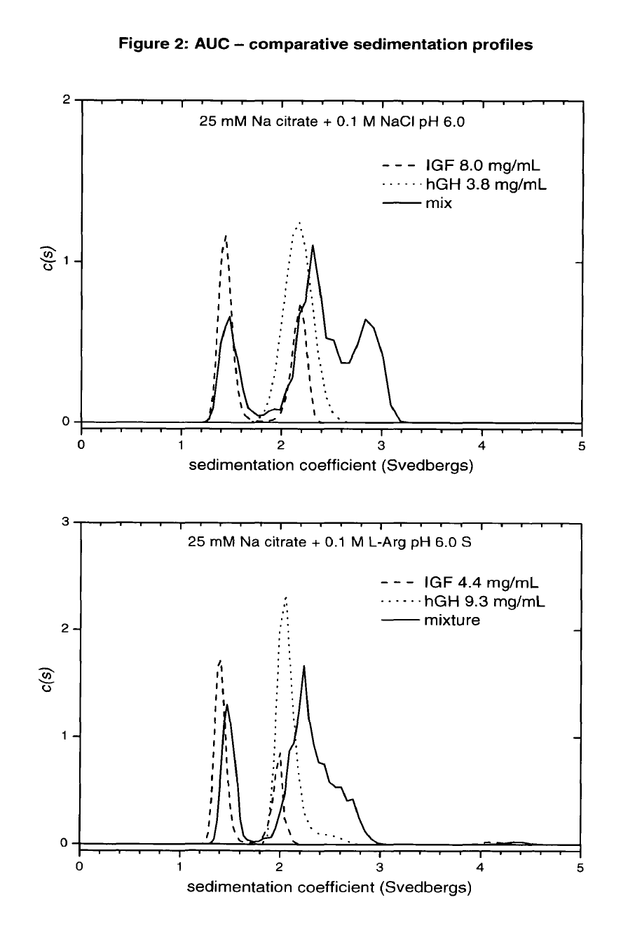

The following definitions are set forth to illustrate and define the meaning and scope of the various terms used to describe the invention herein.

According to the present invention the term “non-aggregating agent” relates to compounds which prevent or reduce formation of insoluble protein aggregates, when proteins are put in a solution.

The term “IGF-1” refers to insulin-like growth factor-1 from any species including but not limited to bovine, ovine, porcine, avian and preferably human in native-sequence or in variant form and from any source, whether natural synthetic or recombinant.

Preferably, IGF-1 is recombinantly produced as e.g. described in U.S. Pat. No. 6,331,414. More preferably, IGF-1 is the active pharmaceutical ingredient in the product commercially marketed as INCRELEX™.

The term “rhIGF-1” refers to recombinant human IGF-1.

The term “GH” refers to growth hormone from any species including but not limited to bovine, ovine, porcine, avian and preferably human in native-sequence or in variant form and from any source, whether natural synthetic or recombinant.

The terms “human growth hormone” and “hGH” relate to human growth hormone produced by methods including natural source extraction and purification, and by recombinant cell culture systems for instance as disclosed in the scientific publication “Direct expression in Escherichia coli of a DNA sequence coding for human growth hormone” Goeddel & al, Nature Vol. 281, October 1979. The sequence of hGH is set forth, for example in Hormone Drugs, Gueriguian et al., USP convention, Rockville, Md. (1982). The terms also cover biologically active human hormone equivalents, e.g., including one or more different amino acid(s) in the overall sequence. Furthermore, the terms as used in this application are intended to cover substitution, deletion and insertion amino acid variants of hGH, i.e., analogs and/or homologs of hGH or hGHs with posttranslational modifications. Two species are often used: the 191 amino acid native species (Somatropin) and the 192 amino acid N-terminal methionine species, both commonly obtained recombinantly.

It is preferred to use methionyl human growth hormone (met-hGH) produced in E. coli, which is sold under the trademark PROTROPIN® by Genentech, Inc. and is identical to the natural polypeptide, with the exception of the presence of an N-terminal methionine residue. Also preferred is the recombinant hGH available from Genentech, Inc. under the trademark NUTROPIN®. More preferred is recombinant rhGH liquid for injection available from Genentech, Inc. under the trademark NUTROPIN AQ®.

The term “buffer” as used herein denotes a pharmaceutically acceptable buffer which preferably confers a pH of 5-6.5. Suitable buffers comprise but are not limited to acetate buffers, citrate buffers, phosphate buffers, succinate buffers and amino acid buffers such as histidine buffers and all salts thereof.

The term “preservative” as used herein means a pharmaceutically acceptable substance to prevent decomposition by microbial growth or by undesirable chemical change.

The terms “surfactant” as used herein means a pharmaceutically acceptable substance to allow dispersion or suspension, by reducing the surface tension of the solvent (such as water) or the interfacial tension between two non miscible liquids. Suitable surfactants are for instance non ionic surfactants such as polysorbates or poloxamers.

The term “bulking agent” as used herein means a pharmaceutically acceptable substance used to increase the amounts of solids and are for instance sucrose, trehalose and mannitol, but not limited to those listed.

The term “tonicity modifer” refers to an isotonic modifier or osmotic adjuster or osmolyte that provides osmolality to the buffer solution. Osmolality refers to the total osmotic activity contributed by ions and nonionized molecules to a solution which includes inorganic salts such as sodium chloride and potassium chloride, polyethylene glycols (PEGs), polypropylene, glycol, glycine, glycerol.

The term “lyophilised” as used herein refers to a formulation that has undergone a process known in the art as freeze-drying, involving freezing the formulation and subsequently removing the ice from the frozen content.

The term “amino acid” as used herein denotes an amino acid (a free amino acid, i.e. not an amino acid in a peptide or protein sequence). An amino acid, as used herein, comprises but is not limited to arginine, glycine, lysine histidine, glutamic acid, aspargic acid, isoleucine, leucine, alanine, phenylalanine, tryprophane, serine, methionine and proline, for instance.

The term “IRF” or “immediate release formulation” refers to a drug composition or mixture of drug compositions, preferably is liquid form, in which there is no carrier that regulates the bioavailability of the drug's active substance to tissues at the site of drug administration in the patient's body.

The term “non-aggregating agent” as used herein refers to a product which prevents the interaction of proteins to form complexes and/or aggregates when they are mixed together in a solution.

In accordance with the present invention, the pharmaceutical composition comprises rhIGF-1 and rhGH and
a non-aggregating agent;
a buffer;
a surfactant;
optionally, a preservative; and
optionally a tonicity modifier or bulking agent.

wherein the non-aggregating agent is present in the composition in a concentration of at least 80 mM.

It is a characteristic of the pharmaceutical composition of the invention that the two active ingredients IGF-1 and GH are present in a single formulation. A “single formulation”, as used herein, is also referred to as a “co-formulation” or a “co-mix”. The terms co-formulation or co-mix are used interchangeably herein.

Preferably, the two active ingredients are human IGF-1 and GH, also called hIGF-1 and hGH herein. It is further preferred that both active ingredients are produced by recombinant means.

In a preferred embodiment, the pharmaceutical composition of the invention is a liquid composition. It is further preferred that it is a multi-dose composition. In the embodiment of a multi-dose composition, a preservative is preferably present.

In a further aspect, the invention relates to processes for the preparation of a pharmaceutical composition comprising IGF-1 and GH. One process according to the invention for the preparation of a pharmaceutical composition may be carried out as follows:
a) Preparing a hGH solution in a buffer at a pH between 5 and 6.5 comprising a non-aggregating agent, a tonicity modifier or bulking agent;
b) Preparing a solution of IGF-1 by dialysing an IGF-1 preparation into the buffer used in step (a) comprising said non-aggregating agent and said tonicity modifier or bulking agent;
c) Adding a surfactant and optionally a preservative to both stock solutions; and
d) Mixing together the solutions of hGH and IGF-1.

In embodiments of this process, in step (a), lyophilized hGH is dissolved in a buffer, or liquid hGH (e.g. approximately 20 mg/ml solution in bicarbonate buffer) is buffer exchanged into another buffer, preferably citrate, succinate or histidine buffer at a convenient pH, preferably between about 5 and 6.5, the buffer containing the non-aggregating agent at a concentration range of between 80 to 200 mM, preferably in the range of between about 100 mM and about 150 mM. Optionally, at least one solution prepared in any of steps (a), (b), (c) or (d) comprises a preservative, preferably phenol or benzyl alcohol.

The term “about”, in the context of amounts of ingredients presented herein, means that the amount can vary by less than ±20% or less than ±15% or less than ±10% or less than ±5%.

In step (b), lyophilized IGF-1 is dissolved into a buffer, or liquid IGF-1 (e.g. approximately 20-35 mg/ml solution in citrate buffer) is buffer exchanged into another buffer, preferably citrate, succinate or histidine at a convenient pH, preferably between about 5 and 6.5, the buffer containing the non-aggregating agent at a concentration range from about 80 mM to about 200 mM.

The two independently prepared solutions are then mixed together.

An alternative process for the preparation of a pharmaceutical composition is also encompassed by the invention.

In accordance with the present invention, the alternative process for the preparation of a pharmaceutical composition of the invention comprises:
a) Preparing a solution I by admixing a buffer, preferably histidine buffer, a non-aggregating agent, preferably arginine, preferably polysorbate 20, optionally a preservative, preferably benzyl alcohol a surfactant, and optionally adjusting the volume with water, the solution I having or being adjusted to a pH of about 5.8;
b) Preparing a solution of IGF-1, in the buffer and non-aggregating agent that are used in step (a), to obtain a solution II;
c) Adding solution II to solution I to obtain a solution III;
d) Preparing a solution IV by admixing a buffer, preferably histidine, a non-aggregating agent, preferably arginine, a surfactant, preferably polysorbate 20, optionally, a preservative, preferably benzyl alcohol, and optionally adjusting the volume with water, the solution IV having or being adjusted to a pH of about 5.8;
e) Preparing a solution of GH in the buffer and non-aggregating agent that are used in step (d), the GH optionally comprising sodium bicarbonate buffer, in order to obtain a solution V;
f) Adding solution V to solution IV to obtain a solution VI;
g) Optionally, independently filtering solutions III and VI;
h) Mixing filtered solutions III and VI at a ratio of IGF-1:GH (w/w) between about 1:1 and 7:1 (w/w), preferably 1.1:1 (w/w), 3.3:1 (w/w) and 6.6:1, to obtain a solution VII; and
i) Optionally, filtering solution VII.

Steps (b) and (e) can e.g. be carried out by diafiltration of a solution comprising IGF-1 or GH into the appropriate buffer and non-aggregating agent or any other suitable solution in order to obtain solutions II and IV.

In an embodiment, solution I and solution IV are identical. In that embodiment, step (d) is obsolete, i.e. solution IV is not prepared. and solution V is simply mixed with solution I to obtain solution VI.

In an embodiment, solutions II and IV may comprise a bulking agent such as e.g. sucrose or mannitol.

In an embodiment, a liquid GH drug substance (i.e. a solution comprising GH, preferably hGH and more preferably rhGH) is directly mixed with solution IV, without any prior buffer exchange or diafiltration into the buffer and non-aggregating agent according to step (e), i.e. without performing step (e) as described above.

Hence, in this embodiment, the process comprises the following steps:
a) Preparing a solution I by admixing a buffer, preferably histidine buffer, a non-aggregating agent, preferably arginine, preferably polysorbate 20, optionally a preservative, preferably benzyl alcohol a surfactant, and optionally adjusting the volume with water, the solution I having or being adjusted to a pH of about 5.8;
b) Preparing a solution of IGF-1, in the buffer and non-aggregating agent that are used in step (a), to obtain a solution II;
c) Adding solution II to solution I to obtain a solution III;
d) Preparing a solution IV by admixing a buffer, preferably histidine, a non-aggregating agent, preferably arginine, a surfactant, preferably polysorbate 20, optionally, a preservative, preferably benzyl alcohol, and optionally adjusting the volume with water, the solution IV having or being adjusted to a pH of about 5.8;
e) variant: Adding a GH drug substance, optionally comprising sodium bicarbonate buffer, to solution IV to obtain a solution VI;
f) Optionally, independently filtering solutions III and VI;
g) Mixing filtered solutions III and VI at a ratio of IGF-1:GH (w/w) between about 1:1 and 7:1 (w/w), preferably 1.1:1 (w/w), 3.3:1 (w/w) and 6.6:1, to obtain a solution VII; and
h) Optionally, filtering solution VII.

In an embodiment of this variant process, solution I and solution IV are identical. In that embodiment, step (d) is obsolete, i.e. solution IV is not prepared, and the GH drug substance is simply mixed with solution I to obtain solution VI.

Preferably, the liquid hGH drug substance is approximately 20 mg/ml hGH solution in bicarbonate buffer of a concentration of about 6-10 mM, preferably 7.5 mM, and is diluted without preliminary diafiltration into a buffer, preferably citrate, succinate or histidine at a convenient pH, preferably between about 5 and 6.2 and optionally containing the non-aggregating agent at a concentration range from about 80 to 200 mM, preferably from about 100 mM or about 150 mM.

In another embodiment, a liquid IGF-1 (e.g. approximately 20-35 mg/ml solution in 200 mM citrate buffer) is buffer exchanged into another buffer, preferably citrate, succinate or histidine buffer at a convenient pH, preferably between about 5 and 6.5 and optionally containing the non-aggregating agent at a concentration range of about 80 to about 200 mM, preferably about 100 mM to about 150 mM. The two independently prepared solutions are then mixed together.

The filtration can be carried out by any suitable means, e.g. cellulose-based filters or PES (polyethersulfone) filters. In a preferred embodiment, filtrations of all solutions (before and after mixing the solutions) may be made by means of 0.22 micrometer filters of low affinity for proteins, such as e.g. polyvinylidene fluoride (PVDF) filters. The membranes of the filters preferably have molecular weight limits of about 5 kDa or about 3 kDa.

Advantageously, the pharmaceutical compositions of the invention are stable for at least one 1 month, 3 months, 6 months, 9 months, a year or up to 2 years.

In a further aspect, the present invention encompasses the use of arginine as a non-aggregating agent in a liquid pharmaceutical composition comprising IGF-1 and GH, preferably hIGF-1 and hGH, more preferably rhIGF-1 and rhGH, wherein the concentration of arginine ranges from about 80 mM to about 200 mM, i.e. is e.g. about 80, about 90 mM, about 100 mM, about 110 mM, about 120 mM, about 130 mM, about 140 mM, about 150 mM, about 160 mM, about 170 mM, about 180 mM, about 190 mM or 200 mM.

It has been found that inclusion of an amino acid in the pharmaceutical composition allows mixtures of IGF-1 and GH to be formulated together in a clear solution formulation, with no loss of visual clarity in the mixture during subsequent refrigeration at 2 to 8° C. for at least 30 days, preferably for at least 6 months, more preferably for at least 12 months.

In a preferred embodiment of the invention, the formulation is stable upon storage at a temperature of −20° C., or between 2° C. and 8° C., for at least 18 months.

In one embodiment the invention encompasses a stable, co-miscible formulation of the active ingredients human Insulin-like growth factor 1 (hIGF-1) and human growth hormone (hGH). In a preferred embodiment, the active ingredients are produced by recombinant means and designated rhIGF-1 and rhGH.

The formulations comprise rhIGF-1 and rhGH, a non-aggregating agent, and a buffer. The formulations may contain a surfactant, preferably a non-ionic surfactant, optionally a preservative, and optionally a tonicity modifier And/or bulking agent.

Preferably, the amino acid which allows mixtures of IGF-1 and GH to be formulated together in a clear solution formulation is arginine or lysine, more preferably arginine (for instance as argininium ion).

Preferably, the amino acid which acts as a non-aggregating agent is added separately to each solution before mixing together in a clear solution formulation. More preferably the final concentration of the a non-aggregating agent in the clear solution is present at a concentration range of about 80 mM to about 200 mM or at a concentration range of about 100 mM to about 180 mM or at a concentration range of about 120 to about 160 mM or at a concentration of about 150 mM.

The pH is adjusted to a value ranging from about 5 to about 7, preferably from about 5.5 to about 6.5, more preferably from about 5.8 to 6.2. In the context of pH values, the term “about” means that the pH value can vary by ±0.2 or ±0.1. The pH of a solution can be adjusted by any suitable means, such as e.g. adding of an appropriate amount of an acidic solution such as e.g. citrate or, preferably, HCl.

The pH to be used in accordance with the present invention can be e.g. 5.1, 5.2, 5.3, 5.4, 5.5, 5.6, 5.7, 5.8, 5.9, 6.0, 6.1, 6.2, 6.3, 6.4, 6.5, preferably is 5.8, 6.2 or about 6.5.

In addition embodiment, the osmolyte or tonicity modifier may be an inorganic salt. If included, the inorganic salt may be e.g. sodium chloride or potassium chloride, preferably sodium chloride, present in the composition at a concentration of 0 to 150 mM, preferably in a concentration of 1 to 50 mM.

In addition, the optional preservative may be selected form the list: phenol, benzyl alcohol, m-cresol, chlorobutanol. Preferred preservatives are phenol or benzyl alcohol. The preservative may be present in the composition at a concentration of about 0.1 to 5% (w/w), preferably about 0.2 to 2% (w/w) or still preferably about 1%.

The surfactant of the composition disclosed in the present invention is e.g. selected from the list: polysorbate (Tween) or a poloxamer such as polysorbate 80, polysorbate 20 or poloxamer 188. Preferably the surfactant is non ionic, more preferably is a polysorbate (Tween) such as polysorbate 80, polysorbate 20 or a poloxamer such as poloxamer 188, more preferably polysorbate 20 or poloxamer 188 in a concentration range from about 0.01 to 3% (w/w) preferably from about 0.03 to 0.50% (w/w) and more preferably of about 0.2% (w/w).

In addition, the buffer may be selected from suitable pharmaceutically acceptable buffers which confer a pH of 5 to 6.5 such as sodium citrate or histidine or both; preferably acetate buffers, citrate buffers, phosphate buffers amino acid such as histidine and all salts thereof, preferred buffers are citrate or histidine. Preferably the buffer is present in the final composition at a concentration between 1 to 100 mM preferably between 1 to 50 mM and more preferably about 10 mM or about 20 mM.

In accordance with the invention the amounts of IGF-1 and GH are about 2 to 40 mg/ml (IGF-1) and about 1 to 12 mg/ml (hGH) respectively, preferred amounts are about 5 to 20 mg/ml (IGF-1) and about 2 to 8 mg/ml (hGH). Further preferred amounts are about 10 mg/ml of IGF-1 and about 3 mg/ml of hGH, or about 13.2 mg/ml of IGF-1 and 2 mg/ml of GH.

The weight ratio of IGF-1:GH (w/w) ranges preferably from 1:1 to 9:1, or alternatively from about 1:9 to 1:1. More preferably the weight ratio of IGF-1:GH (w/w) is selected from the list: 9:1 (w/w); 6:1 (w/w); 3:1 (w/w); 2:1; 3:7 (w/w); 1:1 (w/w); 1:2 (w/w); 1:5 (w/w); 7:3 (w/w); 9:1 (w/w).

More preferred weight ratios of IGF-1:GH (w/w) are selected from 1.1:1, 2.2:1, 3.3:1 and 6.6:1. In an embodiment, the composition comprises a combination of rhIGF-1 and rhGH in a concentration of about 10 to 30 mg/ml (IGF-1) and about 1 to 12 mg/ml (rhGH) respectively and a weight ratio of IGF-1:GH of between about 9:1 and 1:9 (w/w), about 0.01 to 3% (w/w) of a surfactant, optionally about 0.1 to 5% (w/w) of a preservative, about 1 to 150 mM of a buffer preferably citrate or histidine, a non aggregating agent such as arginine or lysine at a concentration range of 80 to 200 mM. Optionally, the composition may also comprise one or two tonicity modifiers such as NaCl, KCl at a concentration of about 0 to 150 mM for NaCl and KCl and/or bulking agents such as trehalose, mannitol, sorbitol or sucrose between 1 to 10% (w/w) of mannitol, sorbitol, trehalose or sucrose.

Furthermore, the invention relates to a process for the preparation of pharmaceutical composition comprising a combination of IGF-1 and GH.

In the pharmaceutical formulations according to the present invention, the human growth hormone and insulin-like growth factor are preferably produced by recombinant means.

In a further embodiment both IGF-1 and GH, preferably in a composition according to the present invention can be administered to the patient, each in effective amounts or each in amounts that are sub-optimal but when combined are effective. Preferably such amounts are about 25 to 250 micrograms IGF-1/kg body weight/day and about 0.05 to 0.5 mg GH/kg body weight/week.

Preferably, the administration of the pharmaceutical formulation is by injection the injection is preferably parenteral such as via the subcutaneous, intramuscular, intravenous or infusion route, the pharmaceutical composition will be used most preferably as daily bolus injection and is preferably an immediate release formulation (IRF).

The patient to be treated is preferably a mammal, in particular human being but it may also be an animal.

In a further embodiment, the invention provides the use of the composition in the manufacture of a medicament for the treatment of a disease characterized by an increase in or control of, the amount of growth hormone in the plasma.

In particular, the invention provides a methods and compositions for the treatment of growth hormone deficiency (GDH); Turner Syndrome, Prader-Willi syndrome (PWS); short stature in children born with very low birth weight (VLBVV), GDH in adults. Also for endocrine disorder for instance comprising administering to a patient suffering from a metabolic disorder characterized by partial endogenous growth hormone activity or signalling an amount of insuline-like growth factor-1 (IGF-1) and an amount of growth hormone (GH) that are effective in combination therapy to improve a metabolic abnormality in the patient. Wherein the patient has adult idiopathic short stature (ISS) syndrome, wherein the patient receives IGF-1 in a single administration per day and receives GH in a single administration per day, and wherein the patient receives the administration of IGF-1 and GH contemporaneously.

The invention also provides methods and compositions for children suffering from growth disorders characterized by partial endogenous growth hormone activity or signalling conditions. These growths which cause disorders in childhood persist into adulthood, and the affected adult can suffer from a variety of metabolic disorders.

According to the present invention hGH and hIGF-1 are used as a medicament or as pharmaceutical composition.

A valuable advantage of the present invention is to provide compositions which may be used as prefilled into a container such as syringes or ready to use formulations.

The following examples serve as illustration of the invention without limiting it.

### EXAMPLE 1

Solubility Tests

Mixtures of INCRELEX® (10 mg/ml solution, formulated in 50 mM Acetate buffer at pH 5.4) and NUTROPIN AQ® (5 mg/ml solution, formulated in 10 mM citrate buffer at pH 6) were prepared at volume ratios ranging from 9:1 to 1:9. The mixtures showed varying degrees of visible precipitation immediately or within a few hours of mixing. Mass spectroscopic analysis of precipitates formed in the NUTROPIN AQ® and INCRELEX® mixtures revealed the presence of both proteins in the precipitates. In table 1 are gathered observations and results relating to the clarity of co-mixtures prepared from commercialized products of IGF-1 (INCRELEX®) and GH (NUTROPIN AQ®).

TABLE 1

| | | | Observation | | |
|---|---|---|---|---|---|
| Ratio | Increlex | Nutropin AQ | Initial | 24 hour | 1 week |
| (v:v) | (mL) | (mL) | (20MAR08) | (21MAR08) | (27MAR08) |
| 9:1 | 3.6 | 0.4 | Very slight free- | Very slight free- | Very slight free- |
| | | | floating | floating | floating |
| | | | precipitate | precipitate | precipitate |
| | | | pH = 5.42 | | |
| 5:1 | 3.6 | 0.72 | Slight free- | Slight free- | Slight free- |
| | | | floating | floating | floating |
| | | | precipitate | precipitate | precipitate |
| | | | pH = 5.51 | | |
| 2:1 | 3.6 | 1.8 | Free-floating | Free-floating | Free-floating |
| | | | precipitate | precipitate | precipitate |
| | | | pH = 5.57 | | |
| 1:1 | 2.0 | 2.0 | Free-floating | Free-floating | Clear with |
| | | | precipitate | precipitate | gelatinous film on |
| | | | pH = 5.64 | | glass |
| 1:2 | 1.8 | 3.6 | Turbid, heavy | Clear with | Clear with |
| | | | precipitate | gelatinous film on | gelatinous film on |
| | | | pH = 5.74 | glass | glass |
| 1:5 | 0.72 | 3.6 | Free-floating | Heavy, chunky | Clear with |
| | | | precipitate | precipitate | gelatinous film on |
| | | | pH = 5.85 | | glass |
| 1:9 | 0.40 | 3.6 | Free-floating | Free-floating | Free-floating |
| | | | precipitate | precipitate | precipitate |
| | | | pH = 5.94 | | |

The solubility of IGF-1 was confirmed to be greater that 20 mg/ml throughout the pH range of the mixtures (5.4-5.9), indicating that the IGF-1 solubility is not causative to the observed precipitate is not IGF-1. The solubility of GH in citrate, acetate or histidine buffers in the pH range was found to be buffer dependent. The results demonstrate a steep decline in solubility of acetate buffered solutions of GH at pH values below 5.6, which may contribute to the observed precipitation in the mixtures which result in the solutions.

However, mixtures of NUTROPIN AQ® with INCRELEX® placebo (which does not contain IGF-1, but is otherwise identical in composition in INCRELEX®), or mixtures of INCRELEX® with the NUTROPIN AQ® placebo, (which does not contain GH, but is otherwise identical in compositions to NUTROPIN AQ®) remain clear in comparison, indicating that the reduced solubility of the proteins may also be related to interaction between the two proteins. Furthermore, INCRELEX® diluted with INCRELEX® placebo to a final concentration of 2.5 mg/mL may be mixed with NUTROPIN AQ® in IGF-1:hGH ratios of 2.2:1 or greater with no precipitation, indicating that the interaction between the proteins is reversible.

### EXAMPLE 2

Comparison and Preparation of Co-Mix Compositions Buffered in Citrate at Various pH

Lyophilized hGH was dissolved in a 10 mM citrate buffer at pH 6, containing 150 mM sodium chloride and 0.2% polysorbate 20, to a final concentration of 5 mg/ml. Solutions of IGF-1 in the different formulation buffers shown in Column 1 of Table 1 were prepared either by dialysis of the IGF-1 into the respective buffer or by reconstitution of lyophilized IGF-1 into the buffer. The final concentration of the IGF-1 solutions prior to mixing with the GH solutions was 10 mg/ml. The GH and IGF-1 solutions were mixed together in the various ratios shown in Table 2.

Visual appearance of co-mixtures prepared from GH in a citrate buffer with IGF-1 in various buffers at pH 5.4 and 6 are gathered in Table 2.

TABLE 2

| | Mixing | Visual appearance of |
|---|---|---|
| | ratio | mixtures after 1-2 Weeks at |
| IGF-1 formulation | hGH:IGF-1 | 5 C. |
| 10 mM Citrate, pH 5.4 | 1:9 | Clear |
| | 3:7 | Clear |
| | 1:1 | Some particulates |
| | 7:3 | Particulates |
| | 9:1 | Cloudy Solution |
| 20 mM Citrate, pH 6 | 1:9 | Clear |
| | 3:7 | Slight particulates |
| | 1:1 | Slight particulates |
| | 7:3 | Slight particulates |
| | 9:1 | Slight particulates on mixing |
| 50 mM Citrate, pH 5.4 | 1:9 | Clear |
| | 3:7 | Clear |
| | 1:1 | Slight particulates |
| | 7:3 | Some particulates |
| | 9:1 | Cloudy Solution |
| 10 mM Acetate, pH 5.4 | 1:9 | Clear |
| | 3:7 | Slightly cloudy, clear after |
| | | mixing |
| | 1:1 | Slightly cloudy solution |
| | 7:3 | Cloudy solution |
| | 9:1 | Cloudy solution |
| 50 mM Acetate, pH 5.4 | 1:9 | Clear |
| | 3:7 | Very slight particulates |
| | 1:1 | Particulates |
| | 7:3 | Cloudy suspension |
| | 9:1 | Cloudy suspension |
| 10 mM Phosphate, pH 6 | 1:9 | Clear after mixing |
| | 3:7 | Cloudy solution |
| | 1:1 | Cloudy solution |
| | 7:3 | Cloudy solution |
| | 9:1 | Cloudy solution |
| 50 mM Phosphate, pH 6 | 1:9 | Slightly cloudy, clear after |
| | | mixing |
| | 3:7 | Cloudy after mixing |
| | 1:1 | Particulates |
| | 7:3 | Cloudy suspension |
| | 9:1 | Cloudy suspension |
| 10 mM Histidine, pH 5.4 | 1:9 | Clear |
| | 3:7 | Slightly cloudy after mixing |
| | 1:1 | Slightly cloudy after mixing |
| | 7:3 | Cloudy solution |
| | 9:1 | Cloudy solution |
| 50 mM Histidine, pH 5.4 | 1:9 | Clear |
| | 3:7 | Mostly clear on mixing |
| | 1:1 | Particulates |
| | 7:3 | Cloudy suspension |
| | 9:1 | Cloudy suspension |
| 10 mM Histidine, pH 6 | 1:9 | Clear |
| | 3:7 | Clear |
| | 1:1 | Slightly cloudy |
| | 7:3 | Slightly cloudy after mixing |
| | 9:1 | Cloudy solution |
| 50 mM Histidine, pH 6 | 1:9 | Clear |
| | 3:7 | Cloudy solution |
| | 1:1 | Cloudy solution |
| | 7:3 | Cloudy solution |
| | 9:1 | Cloudy solution |

The observations recorded in Table 2 show that solutions of IGF-1 in various buffers produce precipitation when mixed with GH formulated in citrate buffer at pH 6.

### EXAMPLE 3

Preparation of Compositions and Tests of Clarity of Citrate Buffered Composition

Approximately 19 mg/ml solutions of each protein (IGF-1 and hGH) were separately dialyzed into a 10 mM citrate buffer at pH 6.0, containing 10 mM arginine. Following overnight dialysis, the solution concentrations were determined by measurement of the ultraviolet (UV) absorbance at 280 nm. The final concentrations of the IGF-1 and hGH solutions were 14 and 21 mg/ml, respectively. Individual aliquots of each solution were constituted with the remaining excipients as shown in Table 3 and diluted to a final protein concentration of 10 mg/ml. Each pair of individually formulated protein solutions (IGF-1 and hGH) were mixed in a 1:1 ratio, to prepare mixtures containing 5 mg/ml of each protein. After the two protein mixtures were prepared, one of the two surfactants was added to both the individual protein formulations and co-mixtures, to the final concentrations. The solutions were inspected after 72 hours of refrigeration. The two mixtures which remained clear at this point (formulation compositions labelled A2 and A10 in Table 3) were stored in the refrigerator and inspected again to confirm that they remained clear after 70 days of storage. In Table 3 are gathered the results of appearance testing of citrate formulations after 72 hours at 5° C.

TABLE 3

| | | | | Co-mix |
|---|---|---|---|---|
| | | | | (5 mg/ml |
| | | | | IGF-1 + |
| | | IGF-1 | hGH | 5 mg/ml |
| | | (10 mg/ml) | (10 mg/ml) | hGH) |
| Sample ID | Excipients | Solution clarity after 72 hours | | |
| A1 | 10 mM Arginine, 0.2% Polysorbate 20, | Yes | No | No |
| | 150 mM NaCl, 1% BzOH, 10 mM Citrate, | | | |
| | pH 6.0 | | | |
| A2 | 100 mM Arginine, 0.2% Polysorbate 20, | Yes | Yes | Yes |
| | 50 mM NaCl, 1% BzOH, 10 mM Citrate, | | | |
| | pH 6.0 | | | |
| A3 | 10 mM Arginine, 0.2% Polysorbate 20, | Yes | No | No |
| | 150 mM NaCl, 0.25% Phenol, 10 mM | | | |
| | Citrate, pH 6.0 | | | |
| A4 | 10 mM Arginine, 0.3% Poloxamer 188, | Yes | No | No |
| | 150 mM NaCl, 1% BzOH, 10 mM Citrate, | | | |
| | pH 6.0 | | | |
| A5 | 100 mM Arginine, 0.3% Poloxamer 188, | Yes | No | No |
| | 150 mM NaCl, 1% BzOH, 10 mM Citrate, | | | |
| | pH 6.0 | | | |
| A6 | 10 mM Arginine, 0.03% Poloxamer 188, | Yes | No | No |
| | 150 mM NaCl, 1% BzOH, 10 mM Citrate, | | | |
| | pH 6.0 | | | |
| A7 | 100 mM Arginine, 0.03% Poloxamer 188, | Yes | No | No |
| | 150 mM NaCl, 1% BzOH, 10 mM Citrate, | | | |
| | pH 6.0 | | | |
| A8 | 10 mM Arginine, 0.3% Poloxamer 188, 75 mM | Yes | No | No |
| | NaCl, 2.5% Mannitol, 1% BzOH, 10 mM | | | |
| | Citrate, pH 6.0 | | | |
| A9 | 10 mM Arginine, 0.3% Poloxamer 188, | Yes | No | No |
| | 5% Mannitol, 1% BzOH, 10 mM Citrate, | | | |
| | pH 6.0 | | | |
| A10 | 100 mM Arginine, 0.3% Poloxamer 188, | Yes | Yes | Yes |
| | 5% Mannitol, 1% BzOH, 10 mM Citrate, | | | |
| | pH 6.0 | | | |

Both of the clear formulations (A2 and A10) contained 100 mM added argininium ion, and little or no added sodium chloride. Two formulations, labeled A1 and A9 in Table 3, which were almost identical in composition to A2 and A10, respectively, but contained a lower amount of added argininium ion (10 mM), did not remain clear.

### EXAMPLE 4

Preparation and Comparative Clarity Test of Histidine Buffered Composition

A 19 mg/ml solution of IGF-1 was dialyzed into a 10 mM Histidine buffer at pH 5.6 containing 10 mM arginine. Following dialysis, the solution concentration was determined by measurement of the ultraviolet (UV) absorbance at 280 nm to be 18 mg/ml. The solution was constituted with appropriate amounts of additional arginine, benzyl alcohol, surfactant (Polysorbate 20 or Poloxamer 188), sodium chloride, and mannitol to prepare the formulation compositions labelled B1 through B8 in Table 4. Aliquots of the IGF-1 only formulations were used to reconstitute lyophilized growth hormone to prepare corresponding formulations containing 5 mg/ml of each protein. The appearance of the solutions was observed after refrigeration at 5° C. for 24 hours. All formulations showed some precipitation at this point, except for the three formulations labelled B3, B4 and B8 in Table 4. These formulations were stored in the refrigerator for a further 65 days, and remained clear at the end of that that time. In Table 4 is gathered histidine buffered formulations at pH 5.6 after 24 hours at 5° C.

TABLE 4

| | | | Co-mix |
|---|---|---|---|
| | | IGF-1 | (5 mg/mL IGF-1 + |
| | | (5 mg/mL) | 5 mg/mL hGH) |
| | | Solution clarity after 72 | |
| Sample ID | Excipients | hours | |
| B1 | 10 mM Arginine, 0.2% Polysorbate 20, 150 mM | Yes | No |
| | NaCl, 1% BzOH, 10 mM Histidine, pH | | |
| | 5.6 | | |
| B2 | 10 mM Arginine, 0.02% Polysorbate 20, 150 mM | Yes | No |
| | NaCl, 1% BzOH, 10 mM Histidine, pH | | |
| | 5.6 | | |
| B3 | 100 mM Arginine, 0.02% Polysorbate 20, 50 mM | Yes | Yes |
| | NaCl, 1% BzOH, 10 mM Histidine, pH | | |
| | 5.6 | | |
| B4 | 100 mM Arginine, 0.3% Poloxamer 188 20, | Yes | Yes |
| | 50 mM NaCl, 1% BzOH, 10 mM Histidine, | | |
| | pH 5.6 | | |
| B5 | 10 mM Arginine, 0.3% Poloxamer 188 20, 150 mM | Yes | No |
| | NaCl, 1% BzOH, 10 mM Histidine, pH | | |
| | 5.6 | | |
| B6 | 10 mM Arginine, 0.03% Poloxamer 188 20, | Yes | No |
| | 150 mM NaCl, 1% BzOH, 10 mM Histidine, | | |
| | pH 5.6 | | |
| B7 | 0.3% Poloxamer 188 20, 5% Mannitol, 1% | Yes | No |
| | BzOH, 10 mM Histidine, pH 5.6 | | |
| B8 | 100 mM Arginine, 0.3% Poloxamer 188, 5% | Yes | Yes |
| | Mannitol, 1% BzOH, 10 mM Histidine, pH | | |
| | 5.6 | | |

All of the three formulation mixtures which remained clear contained 100 mM argininium ion. The formulation mixture labelled B7, which corresponds exactly to the composition of the B8 formulation in Table 4 but without the added arginine, showed precipitation when observed after 24 hours of refrigeration, while the B8 formulation remained clear. Similarly, the formulation mixture labelled as B4 in Table 4 remained clear after prolonged refrigeration, while the B2 formulation, which contained only 10 mM Arginine, and additional sodium chloride, did not remain clear.

### EXAMPLE 5

Preparation of Composition Histidine Buffered at pH 6

Approximately 19 mg/ml solutions of each protein (IGF-1 and hGH) were separately dialyzed into a 10 mM histidine buffer at pH 6 which included 10 mM arginine. Following overnight dialysis, the solution concentrations were determined by measurement of the ultraviolet (UV) absorbance at 280 nm. The final concentrations of the IGF-1 and hGH solutions after dialysis were 11 and 21 mg/ml, respectively. The solutions were individually constituted with appropriate amounts of additional arginine, benzyl alcohol, surfactant (Polysorbate 20 or Poloxamer 188), sodium chloride, and mannitol to prepare the two formulation compositions labelled C1 and C2 in Table 5. The individual protein formulations were mixed in a 1:1 ratio to prepare the co-mixtures, and the appearance of all 6 solutions was observed after refrigeration at 5° C. for 72 hours. Both sets of growth hormone formulations and co-mixtures showed some precipitation, possibly due to the high salt concentration (150 mM) in these formulations. In table 5 are gathered histidine buffered formulation at pH 6 after 72 hours at 5° C.

TABLE 5

| | | | | Co-mix |
|---|---|---|---|---|
| | | | | (5 mg/ml |
| | | IGF-1 | hGH | IGF-1 + |
| | | (10 | (10 mg/ | 5 mg/ml |
| Sample | | mg/mL) | mL) | hGH) |
| ID | Excipients | Solution clarity after 72 hours | | |
| C1 | 100 mM Arginine, 0.3% | Yes | No | No |
| | Poloxamer 188, 150 mM NaCl, | | | |
| | 1% BzOH, 10 mM Histidine, | | | |
| | pH 6.0 | | | |
| C2 | 0.3% Poloxamer 188, | Yes | No | No |
| | 150 mM NaCl, 1% BzOH, | | | |
| | 10 mM Histidine, pH 6.0 | | | |

### EXAMPLE 6

Preparation and Comparison of Citrate and Histidine Buffered Composition

Two formulations were prepared with each protein (IGF-1 and GH), at final protein concentrations of 20 mg/ml (IGF-1) and 6 mg/ml (GH), respectively:
Formulation 1: 10 mM Citrate, 0.2% Polysorbate 20, 1% Benzyl Alcohol, 100 mM Arginine, 50 mM NaCl, pH 6.2
Formulation 2: 10 mM Histidine, 100 mM Arginine, 0.3% Poloxamer 188, 1% Benzyl Alcohol, 50 mM NaCl, pH 5.8

The formulations were prepared by buffer exchange of each protein by tangential flow-filtration, into each of the two buffers (buffer 1 and buffer 2), to prepare four stock solutions at the concentrations shown in Table 6. In Table 6 is gathered the preparation of stock solutions for formulation.

TABLE 6

| | Concentration of | |
|---|---|---|
| | protein after | |
| | buffer exchange (mg/ml) | |
| Buffer System | IGF-1 | GH |
| Buffer 1: 10 mM Citrate, 50 mM NaCl, | 38 mg/ml | 15 mg/ml |
| 100 mM Arginine pH 6.0 | | |
| Buffer 2: 10 mM Histidine, 50 mM NaCl, | 29 mg/ml | 14 mg/ml |
| 100 mM Arginine pH 5.6 | | |

Additional buffer and surfactant stock solutions were added to each stock solution with gentle mixing, followed by addition of the appropriate amount of neat BzOH (benzyl alcohol to achieve the final composition of formulations 1 and 2 with each protein. The protein concentrations of the IGF-1 and hGH formulations were 20 mg/ml and 6 mg/ml, respectively. The four formulations (two IGF-1 formulations and two hGH formulations) were then diluted and/or mixed to achieve the final formulations and co-mixtures in Table 7. The solutions were then stored at 2-8° C. until further dilution/vialing. All protein solutions were sterile filtered using PES membranes and then aliquoted into 3 ml glass vials. The vials were stoppered, crimp-sealed and stored for up to 8 weeks in the refrigerator. The appearance of each solution was evaluated at 2 week intervals for 8 weeks. At the end of 8 weeks, all 14 solutions were still clear and colorless. In table 7 are gathered the results of visual appearance of citrate and histidine formulations containing 100 mM arginine.

TABLE 7

| | | hGH | | Solution appearance | |
|---|---|---|---|---|---|
| | Ratio | (mg/ | IGF-1 | Immediately after | After 8 weeks |
| ID | GH:IGF-1 | ml) | (mg/ml) | mixing | at 5 C. |

Citrate Formulations: 10 mM Citrate, 0.2% Polysorbate 20, 1% Benzyl
Alcohol, 100 mM Arginine, 50 mM NaCl, pH 6.2

| | | | | | |
|---|---|---|---|---|---|
| CA1 | 1:1.1 | 3 | 3.3 | Clear, Colorless | Clear, Colorless |
| | | | | Solution | Solution |
| CA2 | 1:4 | 2.5 | 10 | Clear, Colorless | Clear, Colorless |
| | | | | Solution | Solution |
| CA3 | GH only | 3 | — | Clear, Colorless | Clear, Colorless |
| | | | | Solution | Solution |
| CA4 | IGF-1 | | 10 | Clear, Colorless | Clear, Colorless |
| | only | | | Solution | Solution |
| CC1 | 1:1.1 | 4.5 | 5 | Clear, Colorless | Clear, Colorless |
| | | | | Solution | Solution |
| CC2 | GH only | 6 | — | Clear, Colorless | Clear, Colorless |
| | | | | Solution | Solution |
| CC3 | IGF-1 | — | 20 | Clear, Colorless | Clear, Colorless |
| | only | | | Solution | Solution |

Histidine Formulations: 10 mM Histidine, 100 mM Arginine, 0.3%
Poloxamer 188, 1% Benzyl Alcohol, 50 mM NaCl, pH 5.8

| | | | | | |
|---|---|---|---|---|---|
| HB1 | 1:1.1 | 3 | 3.3 | Clear, Colorless | Clear, Colorless |
| | | | | Solution | Solution |
| HB2 | 1:4 | 2.5 | 10 | Clear, Colorless | Clear, Colorless |
| | | | | Solution | Solution |
| HB3 | GH only | 3 | — | Clear, Colorless | Clear, Colorless |
| | | | | Solution | Solution |
| HB4 | IGF-1 | — | 10 | Clear, Colorless | Clear, Colorless |
| | only | | | Solution | Solution |
| HD1 | 1:1.1 | 4.5 | 5 | Clear, Colorless | Clear, Colorless |
| | | | | Solution | Solution |
| HD2 | GH only | 6 | — | Clear, Colorless | Clear, Colorless |
| | | | | Solution | Solution |
| HD3 | IGF-1 | — | 20 | Clear, Colorless | Clear, Colorless |
| | only | | | Solution | Solution |

The chemical stability of the compositions over the eight week period was verified by periodic analysis of the formulations and co-mixes. They were stored refrigerated at 5° C. and at 25° C., to detect the primary, stability-limiting, degradation products of IGF-1 (des-Gly, Pro-IGF-1) and (deamidated GH) and comparison of the degradation rates with established rates for the registered, long-term stable INCRELEX® (IGF-1, liquid for injection) and NUTROPIN AQ® (GH, liquid for injection) controls. The degradation rates are displayed in Tables 8 and 9. The formulations show no trend for the slow degradation of IGF-1 at 5° C. over the 8 week time period; however, the new formulations and co-mixes stored at 25° C. show stability comparable to typical accelerated degradation rates observed for INCRELEX®. The deamidation of GH in the new formulations and co-mixes shows comparable rates with NUTROPIN AQ® controls, at both 5° C. and at 25° C.

TABLE 8

| | | | | Degradation |
|---|---|---|---|---|
| | | | | rate (% |
| | | | | increase in |
| Formu- | hGH | IGF-1 | | DGP-IGF-1 per |
| lation | (mg/mL | (mg/mL) | Excipients | week) at 25 C. |
| CA1 | 3 | 3.3 | 10 mM Citrate, 0.2% | 1.04E−01 |
| CA2 | 2.5 | 10 | Polysorbate 20, 1% | 1.05E−01 |
| CA4 | | 10 | Benzyl Alcohol, | 9.02E−02 |
| CC1 | 4.5 | 5 | 100 mM Arginine, | 8.49E−02 |
| CC3 | | 20 | 50 mM NaCl, pH 6.2 | 8.96E−02 |
| HB1 | 3 | 3.3 | 10 mM Histidine, | 8.17E−02 |
| HB2 | 2.5 | 10 | 100 mM Arginine, | 8.72E−02 |
| HB4 | | 10 | 0.3% Poloxamer | 7.31E−02 |
| HD1 | 4.5 | 5 | 188, 1% Benzyl | 7.96E−02 |
| HD3 | | 20 | Alcohol, 50 mM | 6.78E−02 |
| | | | NaCl, pH 5.8 | |
| Increlex* | | 10 | 50 mM Acetate, | 1.19E−01 |
| | | | 0.2% Polysorbate | |
| | | | 20, 0.9% Benzyl | |
| | | | Alcohol, 100 mM | |
| | | | NaCl, pH 5.4 | |

*Average degradation rate from 15 lots

TABLE 9

| | | | | Deamidation rate | |
|---|---|---|---|---|---|
| | hGH | IGF-1 | | (% increase per week) | |
| Formulation | (mg/mL | (mg/mL) | Excipients | at 5 C. | at 25 C. |
| CA1 | 3 | 3.3 | 10 mM Citrate, 0.2% | 1.91E−02 | 2.85E−01 |
| CA2 | 2.5 | 10 | Polysorbate 20, 1% | 2.09E−02 | 3.25E−01 |
| CA3 | 3 | | Benzyl Alcohol, | 1.56E−02 | 2.71E−01 |
| CC1 | 4.5 | 5 | 100 mM Arginine, 50 mM | 2.48E−02 | 3.04E−01 |
| CC2 | 6 | | NaCl, pH 6.2 | 2.36E−02 | 3.00E−01 |
| HB1 | 3 | 3.3 | 10 mM Histidine, | 1.53E−02 | 1.33E−01 |
| HB2 | 2.5 | 10 | 100 mM Arginine, 0.3% | 2.01E−02 | 1.62E−01 |
| HB3 | 3 | | Poloxamer 188, 1% | 1.43E−02 | 1.22E−01 |
| HD1 | 4.5 | 5 | Benzyl Alcohol, 50 mM | 1.66E−02 | 1.51E−01 |
| HD2 | 6 | | NaCl, pH 5.8 | 1.99E−02 | 1.44E−01 |
| NUTROPIN | 5 | | 10 mM Citrate, 0.2% | 2.12E−02 | 2.99E−01 |
| AQ ® | | | Polysorbate 20, 0.25% | | |
| | | | Phenol, 150 mM NaCl, | | |
| | | | pH 6.0 | | |

### EXAMPLE 7

Preparation and Comparison of Histidine Buffered Compositions of IGF-1

Two formulations were prepared with IGF-1 at a final protein concentrations of 20 mg/ml (IGF-1):
Formulation 1: 20 mM Histidine, 0.2% Polysorbate 20, 1% Benzyl Alcohol, 150 mM Arginine, pH 5.8
Formulation 2: 50 mM Histidine, 0.2% Polysorbate 20, 1% Benzyl Alcohol, 150 mM Arginine, pH 5.8

The formulations were prepared by buffer exchange of protein into each of the two buffers, formulated by addition of surfactant and preservative, and evaluated for stability alongside INCRELEX® controls. The stability data at 5° C., 25° C. and 40° C. presented in Table 10.

TABLE 10

| Peak/ | | Degradation rate (Day-1) | | |
|---|---|---|---|---|
| Group | Sample | 5 C. | 25 C. | 40 C. |
| % Active | 50 mM Histidine | 1.43E−02 | 4.00E−02 | 1.30E−01 |
| 1 | formulation | | | |
| | Increlex Control | 1.86E−02 | 3.71E−02 | 1.43E−01 |
| % Main | 50 mM Histidine | −2.14E−02 | −5.90E−02 | −2.43E−01 |
| Peak | formulation | | | |
| | Increlex Control | −2.90E−03 | −3.29E−02 | −2.10E−01 |
| % Active | 20 mM Histidine | 1.72E−04 | 1.87E−02 | 1.46E−01 |
| 1 | formulation | | | |
| | Increlex Control | 2.67E−03 | 1.66E−02 | 1.39E−01 |
| % Main | 20 mM Histidine | −1.36E−02 | −5.87E−02 | −3.03E−01 |
| Peak | formulation | | | |
| | Increlex Control | 4.39E−03 | −1.75E−02 | −2.12E−01 |

### EXAMPLE 8

Stabilities Data on Combo Formulations

TABLE 11

| | | | | Increlex | | rhIGF-1 | | Nutropin | | rhGH Only | | | | | |
|---|---|---|---|---|---|---|---|---|---|---|---|---|---|---|---|
| | | | | (% | | Only (% | | (% | | (% | | 1:2.2 Co-form. | | 1:6.6 Co-form. | |
| | | | Actual | Monomer) | | Monomer) | | Monomer) | | Monomer) | | (% Monomer) | | (% Monomer) | |
| Temp | Months | Test/Pull Date¹ | Months | IGF-1 | GH | IGF-1 | GH | IGF-1 | GH | IGF-1 | GH | IGF-1 | GH | IGF-1 | GH |
| n/a | 0 | 22Oct09 | 0.000 | 99.5 | — | 99.7 | — | — | 99.9 | — | 100.0 | 99.5 | 100.0 | 99.6 | 100.0 |
| 5° C. | 3 | 27Jan10 | 3.189 | 99.3 | — | 99.5 | — | — | 99.6 | — | 99.9 | 99.5 | 100.0 | 99.6 | 100.0 |
| | 6 | 03May10 | 6.345 | 99.9 | — | 99.9 | — | — | 99.5 | — | 100.0 | 100.0 | 100.0 | 100.0 | 100.0 |
| 15° C. | 1 | 18Nov09 | 0.888 | 99.6 | — | NT | NT | — | 99.5 | NT | NT | 99.4 | 100.0 | 99.5 | 100.0 |
| | 3 | 18Jan10 | 2.893 | 99.4 | — | NT | NT | — | 99.4 | — | 99.8 | 99.4 | 99.9 | 99.4 | 100.0 |
| | 6 | 16Apr10 | 5.786 | 99.9 | — | NT | NT | — | 99.1 | — | 99.9 | 100.0 | 100.0 | 100.0 | 100.0 |
| 25° C. | 1 | 18Nov09 | 0.888 | 99.5 | — | 99.5 | — | — | 99.7 | NT | NT | 99.2 | 100.0 | 99.3 | 100.0 |
| | 3 | 18Jan10 | 2.893 | 98.9 | — | 98.8 | — | — | 98.5 | — | 98.8 | 99.0 | 99.4 | 99.2 | 100.0 |
| | 6 | 16Apr10 | 5.786 | 99.2 | — | 98.4 | — | — | 99.7 | NT | NT | 97.9 | 98.0 | 98.6 | 98.9 |
| 40° C. | 1 | 18Nov09 | 0.888 | 97.7 | — | 95.0 | — | — | 98.0 | — | | 98.0 | 93.3 | 97.6 | 94.5 |
| | 3 | 18Jan10 | 2.893 | 92.3 | — | 83.2 | — | — | 91.9 | — | | 94.3 | 92.9 | 93.9 | 88.3 |

¹Since samples are stores at 5° C. until testing, the test date is used to trend 5° C. data and the pull date is used to trend accelerated stability data for greatest accuracy.
NT: Sample was not tested

These data have been produce by size exclusion chromatography and provide 1, 3 and 6 months stabilities.

### EXAMPLE 9

Establishment of an Alternative Co-Formulation Process of rhIGF-1 and rhGH

1. Materials and Method

1.1. Raw Materials

The following raw materials were used in the study and depicted in Table 12:

12 - Raw materials

| Material | Supplier |
|---|---|
| rhGH | Genentech |
| rhIGF-1 | Lonza |
| Citric acid | Sigma |
| Arginine-HCl | Merck |
| Phenol | Merck |
| Poloxamer 188 | BASF |
| Sucrose | Beghin Say |
| Sodium hydroxide | Merck |
| WFI | Cooper |
| Vials 5 mL VB type lyo | Schott |
| Stoppers 13 mm | West - CTSU |
| Crimps alu 13 mm | West |

1.2. Equipment

The following equipments were employed used for the study:
Autoclave FEDEGARI,
Bottles sterile polyethylene (PE) Nalgene ref. 2019,
Cassettes BIOMEX® PES ref.: PXB005A50 for diafiltration,
Clean room, laminar flow hood,
Cogent μScale MILLIPORE tangential flow filtration equipment,
Filters MILLEX® (33 mm) PES 0.22 μm (MILLIPORE),
Filters MILLEX® (33 mm) PVDF 0.22 μm (MILLIPORE),
Glass beakers,
Graduated cylinders,
Magnetic stirrers,
Micropipettes P1000,
Oven FEDEGARI,
Pharmacopoeia compliant vial observation equipment,
Pump FLEXICON PF6 n° 212118,
Syringe PE 50 mL,
Test tubes Eppendorf 1.5 mL,
Test tubes Falcon 50 mL and 15 mL,
Tygon tubes (1.6 mm+dedicated needle)
Washing machine CORIMA.

2. Process and Formula Composition Optimization

A process and formulation optimization trial study was performed. The formulation compositions were as described in Table 13 below:

13 - Composition of prototype formulations A3-c

| | | |
|---|---|---|
| RATIO | 2.2:1 | 2.2:1 |
| NAME | A3 | A3-c |
| Process strategy | A | A |
| rhIGF-1 [mg/mL] | 7.9 | 7.9 |
| rhGH [mg/mL] | 3.6 | 3.6 |
| pH | 6.0 | 6.0 |
| Bulking agent [mM] | Sucrose 200 | Sucrose 140 |
| Arginine HCl [mM] | 150 | 150 |
| Histidine [mM] | — | — |
| Citrate [mM] | 20 | 20 |
| Succinate [mM] | — | — |
| Poloxamer 188 | 2 | 2 |
| [mg/mL] | | |
| Polysorbate 20 | — | — |
| mg/mL] | | |
| Benzyl alcohol | — | — |
| [mg/mL] | | |
| Phenol [mg/mL] | 3.7 | 3.7 |
| Anti-oxidant [mM] | — | — |

2.1. rhIGF-1 Tangential Flow Filtration (TFF) Process

The rhIGF-1 TFF process was carried out with using the following parameters:
IGF-1 quantity to engage:
   80 mL of rhIGF-1 at 25 mg/mL,
diafiltration concentration 25 mg/mL,
exchange buffer: 20 mM citrate, 150 mM Arg. pH 6.0,
PELLICON® XL cassette: regenerated cellulose membrane, 5 KDa kDa cut-off,
TMP 18-22 atm,
Pump set at 12% of capacity,
6 diavolumes,
rhIGF-1 final concentration 30 mg/mL.

2.2. Formulation of the Proteins:
1. In this alternative process, all the excipients were first mixed together and then the protein was added to the excipients.
2. The solution of excipients was prepared at a pH lower than 6.0, so that the addition of the GH DS (at a pH around 7.5-8) would end up by giving a solution at pH lower than 7.

2.2.1. IGF-1 Formulation
1. Weighed 8.44% of final volume of citrate 80 mM/arginine 600 pH 5.5 in a glass beaker.

2. Added 10% of final volume of phenol 3.7%.
3. Added 10% of final volume of poloxamer 188 2%.
4. Mixed to homogenization.
5. Added 3.84 g of sucrose.
6. Mixed to solubilization and homogenization.
7. pH value measured: 5.86.
8. While the solution was gently stirred, the volume corresponding to 1.6 g of diafiltered rhIGF-1 was added. The volume was calculated as follows:
9. 52.7 (mL) (Volume of diafiltered IGF-1)=1600 [mg] (quantity of rhIGF-1 necessary)/30.36 [mg/mL] (IGF-1 concentration)
10. Mixed to homogenization.
11. pH value measured: 6.05, therefore no pH correction considered.
12. Brought to final volume of 80 mL with WFI.
13. pH value measured: 6.05.
14. Filtration with 0.22 μm PES filters.
15. Filtration with 0.22 μm PVDF filters.

2.2.2. Formulation of GH
1. Added 25% of final volume of citrate 80 mM/arginine 600 pH 5.5 in a glass beaker.
2. Added 10% of final volume of phenol 3.7%.
3. Added 10% of final volume of poloxamer 188 2%.
4. Added 10% of final volume of WFI.
5. Mixed to homogenization.
   a. Limpid with few particles (probably of environmental origin),
   b. pH value measured: 5.9.
6. Added 4.83 g of sucrose.
7. Mixed to solubilization and homogenization.
   a. pH: 5.87;
8. Added 30 mL of GH DS while the solution is stirred gently.
9. Mixed to homogenization.
   a. pH value measured: 6.6.
   b. Final pH 6.0.
10. Filtration with 0.22 μm PES filters.
11. Filtration with 0.22 μm PVDF filters.

2.2.3 Co-Formulation (or Co-Mix)

To make 45 mL of final co-mix rhIGF-1/rhGH (2.2:1):
1. Added 17.9 mL of IGF-1 formulation to 27.1 mL of rhGH under magnetic stirring.
2. Mixed to homogenization.
3. Solution appeared limpid and without visible particles.
4. Filtration with 0.22 μm PES filters in the clean room of building 2: obtained limpid solution without visible particles/easy to withdraw carry out by syringe.

3. Application of the Alternative Process to a Selected Formulation Using Histidine as a Buffer

3.1. Formulation Feasibility

The formulation was carried out as described in §2.2.1 for rhIGF-1 formulation, §2.2.2 for rhGH formulation and §2.2.3 for co-formulations 2.2, 1 Co-mix except for the use of:
histidine instead of citrate as the buffering agent,
pH 6.0 instead of 5.8,
polysorbate-20 instead of poloxamer-188 as a surfactant,
pure benzyl alcohol instead of a 10% phenol solution as a preservative,
2.5% solution of HCl instead of 2.5% solution of citric acid to correct the pH,
PVDF filters to filter the stand-alone protein products and the co-mix formulation,
Co-formulations with ratios 1.1:1 and 6.6:1 were prepared instead of the co-formulation mix 2.2:1.

3.2 Optimization of the Filtration-Replicability

The process was carried out as described already in §3.1 (in a lab at 21° C.), except for the filtration process that was carried out according to the sequences below:
150 mL of rhGH formulation subjected to a 1^{st} clarifying filtration with PES 0.22 μm+2^{nd} sterilizing filtration with PVDF.

150 mL of rhGH formulation subjected to a 1^{st} clarifying filtration with PVDF 0.22 μm+2^{nd} sterilizing filtration with PVDF.

The following observations were made:
The rhGH solution before clarifying filtrations appeared as mildly opalescent and free of precipitates.
After both filtration series, the rhGH formulations appeared free of particles.
Visual inspections analyses confirmed the positive effect of using PVDF filters for final sterilizing filtration of formulations and co-formulations, i.e. the strong reduction of visible particles and the good stability when this type of filter is used.

TABLE 14
Formulation and co-formulation compositions

| | | Compositions | | | |
|---|---|---|---|---|---|
| | | | | Co- | Co- |
| | | | | formulation | formulation |
| | | Individual GH | Individual IGF-1 | Ratio IGF-1/GH | Ratio IGF-1/GH |
| | | formulation | formulation | 1.1:1 | 6.6:1 |
| pH | | 5.8 | | | |
| Buffer | Histidine | 20 mM | | | |
| | Na | 7.5 mM | — | 5.6 mM | 2.5 mM |
| | bicarbonate | | | | |

| | | |
|---|---|---|
| Surfactant | Polysorbate | 0.2% |
| | 20 | |
| Preservative | Benzyl alcohol | 1% |
| Stabilizing | Arginine | 150 mM |
| agent | | |

| | | | |
|---|---|---|---|
| 6 mg/mL | 20 mg/mL | 5:4.5 | 13.2:2 |
| | | (mg:mg)/mL | (mg:mg)/mL |

### EXAMPLE 10

Preparation Processes for a rhIGF-1 and rhGH Co-Formulation

rhGH bulk (drug substance or DS) is a 20 mg/ml solution in bicarbonate buffer of concentration 7.5 mM. rhIGF-1 bulk (DS) is a 25-35 mg/ml solution in 200 mM citrate buffer.

3 different types of processes were prepared as follows (in the following designated process I, and alternative processes A and B):

Steps of Process I:
rhGH and rhIGF-1 diafiltration for buffer exchange (original DS buffer vs. Histidine (His) 20 mM/Arginine (Arg) 150 mM pH 5.8),
for each individual protein, compounding was performed in the following order of introduction:
   addition of exchange buffer to adjust concentration of DS,
   addition of the solution of polysorbate PS 20,
   addition of the solution of benzyl alcohol (BA),
co-mixing of individual formulations to produce a stable co-formulation.

Steps of Process A:
rhGH and rhIGF-1 diafiltration for buffer exchange (original DS buffer vs. Histidine (His) 20 mM/Arginine (Arg) 150 mM pH 5.8),
final compounding for each protein was carried out in the following order of introduction
   concentrated buffer,
   solution of surfactant,
   solution of preservative,
   WFI (water for injection),
   bulking agent (if any),
   diafiltered drug substance,
   pH correction with citric acid (or HCl),
   WFI to final volume.
co-mixing of individual formulations to produce a stable co-formulation.

Steps of Process B:
rhIGF-1 diafiltration for buffer exchange (original DS buffer vs. Histidine (His) 20 mM/Arginine (Arg) 150 mM pH 5.8),
no diafiltration of rhGH bulk, but direct formulation of rhGH bulk (i.e. addition of rhGH DS to excipients mixture without buffer exchange).
Final compounding was carried out following the same order of introduction of components as in process A.
co-mixing of individual formulations to produce a stable co-formulation.

1. A pharmaceutical composition comprising: IGF-1; GH; a non-aggregating agent; a buffer; and a surfactant; wherein the non-aggregating agent is present in the composition at a concentration of at least about 80 mM.

2. The composition of claim 1, wherein the non-aggregating agent is arginine or lysine.

3. The composition of claim 2, wherein the non-aggregating agent is arginine and is present at a concentration of from about 80 mM to about 200 mM.

4. The composition of claim 1, wherein the buffer is histidine, succinate or citrate and is present at a concentration of from about 1 to 50 mM.

5. The composition of claim 1, wherein the surfactant is a non-ionic surfactant.

6. The composition of claim 5, wherein the non-ionic surfactant is polysorbate 20 and is present at a concentration of from about 0.1 to 0.3% (w/w).

7. The composition of claim 5, wherein the non-ionic surfactant is poloxamer 188 and is present at a concentration of from about 0.1 to 0.5% (w/w).

8. The composition of claim 1, further comprising a tonicity modifier that is sodium chloride.

9. The composition of claim 1, further comprising a bulking agent that is mannitol and/or sucrose.

10. The composition of claim 8, wherein sodium chloride is present at a concentration of from about 1 to about 50 mM.

11. The composition of claim 1, further comprising a preservative that is benzyl alcohol or phenol.

12. The composition of claim 10, wherein the preservative is benzyl alcohol and is present at a concentration of from about 0.2 to about 2 (w/w).

13. The composition of claim 1, wherein the weight ratio of IGF-1:GH (w/w) is from about 1:1 to 1:9 (w/w).

14. The composition of claim 1, wherein the weight ratio of IGF-1:GH (w/w) is from about 1:1 (w/w) to 7:1 (w/w).

15. The composition of claim 1, having a pH of from about 5.0 to about 6.5.

16. (canceled)

17. A process for the preparation of a pharmaceutical composition, the process comprising:
(a) preparing a hGH solution comprising a non-aggregating agent and a tonicity modifier or bulking agent in a buffer at a pH between 5 and 6.5;
(b) preparing an IGF-1 solution by dialysing an IGF-1 preparation into a solution comprising the non-aggregating agent, tonicity modifier, and buffer used in step (a);
(c) adding a surfactant to both hGH and IGF-1 solutions; and
(d) mixing the solutions.

18. The process of claim 17, wherein the non-aggregating agent is arginine or lysine.

19. A process for the preparation of a pharmaceutical composition, the process comprising:
(a) preparing a first solution by admixing a buffer, a non aggregating agent, and a surfactant, the solution having or being adjusted to a pH of about 5.8;
(b) preparing an second solution comprising IGF-1 using the same buffer and non-aggregating agent as used in step (a);
(c) adding the second solution to the first solution to produce a third solution;
(d) preparing a fourth solution by admixing a buffer, a non-aggregating agent, and a surfactant, the solution having or being adjusted to a pH of about 5.8;
(e) adding a fifth solution comprising GH to the fourth solution to produce a sixth solution; and
(f) mixing the third and sixth solutions at a ratio of IGF-1:GH (w/w) between about 1:1 and 7:1 (w/w) to obtain a seventh solution.

20. (canceled)

21. The process of claim 19, wherein the solution comprising GH further comprises a sodium bicarbonate buffer.

22. The process of claim 18, further comprising the step of filtering the seventh solution using polyvinylidene fluoride filters.

23. (canceled)

24. (canceled)
